(19) United States
(12) Patent Application Publication
(10) Pub. No.: US 2025/0152906 A1
(43) Pub. Date: May 15, 2025

(54) SURGICAL GAS DELIVERY SYSTEM FOR GAS SEALED INSUFFLATION AND RECIRCULATION WITH PNEUMATICALLY POWERED BLOCKING VALVES

(71) Applicant: Conmed Corporation, Largo, FL (US)

(72) Inventor: Michael L. Koltz, Aurora, CO (US)

(73) Assignee: Conmed Corporation, Largo, FL (US)

(21) Appl. No.: 18/839,786

(22) PCT Filed: Mar. 8, 2023
(86) PCT No.: PCT/US2023/014839

§ 371 Date: Aug. 20, 2024

Related U.S. Application Data
(60) Provisional application No. 63/319,562, filed on Mar. 14, 2022.

(51) Int. Cl. A61M 16/20

(52) U.S. Cl. CPC A61M 16/207; A61M 16/202

(57) ABSTRACT

A blocking valve assembly for a surgical gas delivery device is disclosed, which includes a valve body supporting a plurality of pneumatically actuated pistons, each piston mounted for independent movement within a respective piston cylinder between a first position blocking a respective gas passage communicating with a filter cartridge to facilitate a leak test of the gas delivery device and a second position permitting gas flow through the gas passage communicating with the filter cartridge to facilitate a surgical procedure.

### CROSS-REFERENCE TO RELATED APPLICATIONS

The subject application claims the benefit of priority to U.S. Provisional Patent Application No. 63/319,562 filed Mar. 14, 2022, the disclosure of which is incorporated herein by reference in its entirety.

### BACKGROUND OF THE DISCLOSURE

### 1. Field of the Disclosure

The subject disclosure is directed to endoscopic surgery, and more particularly, to a surgical gas delivery system for gas sealed insufflation and recirculation, which includes pneumatically controlled blocking valves for use during a self-testing sequence prior to an endoscopic or laparoscopic surgical procedure.

### 2. Description of Related Art

Laparoscopic or “minimally invasive” surgical techniques are becoming commonplace in the performance of procedures such as cholecystectomies, appendectomies, hernia repair and nephrectomies. Benefits of such procedures include reduced trauma to the patient, reduced opportunity for infection, and decreased recovery time. Such procedures within the abdominal (peritoneal) cavity are typically performed through a device known as a trocar or cannula, which facilitates the introduction of laparoscopic instruments into the abdominal cavity of a patient.

Additionally, such procedures commonly involve filling or “insufflating” the abdominal cavity with a pressurized fluid, such as carbon dioxide, to create an operating space, which is referred to as a pneumoperitoneum. The insufflation can be carried out by a surgical access device, such as a trocar, equipped to deliver insufflation fluid, or by a separate insufflation device, such as an insufflation (veress) needle. Introduction of surgical instruments into the pneumoperitoneum without a substantial loss of insufflation gas is desirable, to maintain the pneumoperitoneum.

During typical laparoscopic procedures, a surgeon makes three to four small incisions, usually no larger than about twelve millimeters each, which are typically made with the surgical access devices themselves, often using a separate inserter or obturator placed therein. Following insertion, the obturator is removed and the trocar allows access for instruments to be inserted into the abdominal cavity. Typical trocars provide a pathway to insufflate the abdominal cavity, so that a surgeon has an open space in which to work.

The trocar must also provide a way to maintain the pressure within the cavity by sealing between the trocar and the surgical instrument being used, while still allowing at least a minimum amount of freedom of movement for the surgical instruments. Such instruments can include, for example, scissors, grasping instruments, and occluding instruments, cauterizing units, cameras, light sources and other surgical instruments. Mechanical sealing elements are typically provided on trocars to prevent the escape of insufflation gas from the abdominal cavity. These sealing mechanisms often comprise a duckbill-type valve or wiper seal made of a relatively pliable material, to seal around an outer surface of surgical instruments passing through the trocar.

SurgiQuest, Inc., a wholly owned subsidiary of ConMed Corporation has developed unique gas sealed surgical access devices that permit ready access to an insufflated surgical cavity without the need for conventional mechanical valve seals, as described, for example, in U.S. Pat. Nos. 7,854,724 and 8,795,223, which are incorporated herein by reference. These surgical access devices are constructed from several nested components including an inner tubular body portion and a coaxial outer tubular body portion. The inner tubular body portion defines a central lumen for introducing conventional laparoscopic surgical instruments to the abdominal cavity of a patient and the outer tubular body portion defines an annular lumen surrounding the inner tubular body portion for delivering insufflation gas to the abdominal cavity of the patient and for facilitating periodic sensing of abdominal pressure.

SurgiQuest, Inc. has also developed a multi-modal surgical gas delivery system to provide functionality to its gas sealed surgical access devices, as described for example in U.S. Pat. No. 9,199,047, which is incorporated herein by reference in its entirety. The functionalities provided by the gas delivery system include insufflation, smoke evacuation, recirculation and filtration of surgical gases.

The gas delivery device disclosed in U.S. Pat. No. 9,199,047 is configured to perform a self-test for leaks in the system prior to a surgical procedure. For the self-test, two prerequisites are necessary. The gas supply must be connected to the system, and there can be no filter cartridge seated in the filter interface of the gas delivery device. If a filter cartridge is seated in the device or the gas supply is empty, the device will provide an error message. When a filter cartridge is inserted into the filter interface and the latching mechanism is rotated to move the filter cartridge into a seated position, spring-loaded blocking valves associated with a pressure line and a suction line of the gas delivery device are moved from a blocking position to permit gas to flow from the gas delivery device to the surgical access device through the filter cartridge.

In the self-test mode, the spring-loaded blocking valves for the gas recirculation circuit are closed. At this time, the delivery device will test for leakage in the system, compressor function and all valve functions. If the self-test succeeds, the device starts in a normal mode. Otherwise, an error message is shown on the user interface screen of the device.

It would be beneficial to automate the blocking valves by way of pneumatic power and electrical control circuits so as to enable system leak self-testing with the filter cartridge installed. This would preclude the need for a user to remove the filter cartridge from the filter interface to perform a system self-test. Additionally, independent pneumatic actuation of the blocking valves would reduce the amount of energy a user needs to expend to latch the filter cartridge in the interface of the delivery device, since the spring-load on the mechanical blocking valves would not need to be overcome by the user.

The reduced energy equates to reduced force and/or torque imparted by the user and enables reduction in the size of the latching mechanism, including the length of the lever. With a smaller latching mechanism, the overall size and weight of the gas delivery device can be reduced, thereby improving operating room workflow and reducing the potential for user fatigue and injury during relocation and repositioning of equipment.

Furthermore, the independent translation of each pneumatically actuated blocking valve would preclude the need for the face seals of the filter cartridge to be coplanar (see e.g., commonly assigned U.S. Pat. No. 8,974,569, incorporated herein by reference), which enables increased tolerance of normal manufacturing variation within the filter cartridge components.

### SUMMARY OF THE DISCLOSURE

The subject disclosure is directed to a new and useful blocking valve assembly for a multi-modal surgical gas delivery device, which includes a valve body supporting a plurality of pneumatically actuated pistons. Each pneumatically actuated piston is mounted for independent movement within a respective piston cylinder between a first position blocking a respective gas passage communicating with a filter cartridge to facilitate a leak test of the gas delivery device and a second position permitting gas flow through the gas passage communicating with the filter cartridge to facilitate a surgical procedure. Preferably, the first position of the pneumatically actuated piston is a retracted position within the piston cylinder and the second position of the pneumatically actuated piston is an extended position within the piston cylinder. The gas passage is preferably defined at least in part by a central lumen extending through each pneumatically actuated piston. A stationary plug is positioned within each piston cylinder for sealing the central lumen of the pneumatically actuated piston when the piston is in the retracted position. In an embodiment of the subject disclosure, each pneumatically actuated piston is part of a two-stage piston arrangement, which includes a proximal drive piston for increasing compression force relative to the filter cartridge.

The blocking valve assembly further includes a control valve in fluid communication with each piston cylinder of the valve body for pneumatically actuating each piston to move each piston from the first position toward the second positon. Preferably, the control valve is a vented solenoid valve, but other types of control valve may be employed including for example a proportional control valve in the form of a motor actuated drive screw.

Each pneumatically actuated piston is biased into the retracted position by a respective coiled return spring. And each pneumatically actuated piston is adapted and configured to move toward the extended position, against the bias of the return spring, when pneumatic pressure in the respective piston cylinder exceeds approximately 2 bar.

The plurality of pneumatically actuated pistons supported in the valve body includes a first piston corresponding to a gas supply passage of the filter cartridge, a second piston corresponding to a gas return passage of the filter cartridge and a third piston corresponding to an insufflation passage of the filter cartridge.

The subject disclosure is also directed to a new and useful surgical gas delivery device, which includes a device housing defining a reception cavity configured to receive a filter cartridge having a plurality of gas passages extending therethrough, and a valve body located within the device housing adjacent the reception cavity and supporting a plurality of pneumatically actuated pistons. Each piston is mounted for independent movement within a respective piston cylinder of the valve body between a retracted position blocking a respective gas passage communicating with a filter cartridge to facilitate a leak test of the gas delivery device and an extended position permitting gas flow through the gas passage communicating with the filter cartridge to facilitate a surgical procedure.

The surgical gas delivery device further includes a control valve located within the device housing and in fluid communication with each piston cylinder of the valve body for pneumatically actuating each piston. A driver is provided in the device housing for activating the control valve to deliver pressurized gas to the valve body to move each of the pneumatically actuated pistons to the extended position, and a processor is provided for commanding the driver to activate the control valve.

These and other features of the subject disclosure will become more readily apparent to those having ordinary skill in the art to which the subject disclosure appertains from the detailed description of the embodiments taken in conjunction with the following brief description of the drawings.

### BRIEF DESCRIPTION OF THE DRAWINGS

So that those skilled in the art will readily understand how to make and use the gas delivery system of the subject disclosure without undue experimentation, preferred embodiments thereof will be described in detail herein below with reference to the figures wherein:

FIG. 1 is a system level schematic diagram of the surgical gas delivery device with the blocking valve assembly of the subject disclosure in conjunction with a gas sealed surgical access device;

FIG. 2 a perspective view of a multi-modal surgical gas delivery device including a graphical user display and an interface having a reception cavity for a disposable filter cartridge;

FIG. 3 is a perspective view of the main valve body of the blocking valve assembly of the subject disclosure, showing three piston sealing faces;

FIG. 4 is a perspective view of the pneumatically actuated blocking valve assembly of the subject disclosure, which is installed in the gas delivery device of FIG. 1 and includes the main valve body of FIG. 3 adjacent the interface of a gas delivery device, and a vented solenoid valve associated therewith;

FIG. 5 is a cross-sectional view of the main valve body of the blocking valve assembly taken along line 5-5 of FIG. 4 positioned adjacent the interface of the gas delivery device, with a filter cartridge seated in the reception cavity, wherein a pneumatically actuated piston is shown in a partially extended operational position;

FIG. 6 is a cross-sectional view of the main valve body of the blocking valve assembly of FIG. 4 with a pneumatically actuated piston shown in a fully retracted blocking position;

FIG. 7 is a cross-sectional view of the main valve body of the blocking valve assembly of FIG. 4 with a pneumatically actuated piston shown in a fully extended position, in the absence of a filter cartridge; and

FIG. 8 is a cross-sectional view of another embodiment of the valve body of the blocking valve assembly of the subject disclosure, which includes pneumatically actuated two-stage pistons shown here in a fully extended position, in the absence of a filter cartridge.

### DETAILED DESCRIPTION OF THE EMBODIMENTS

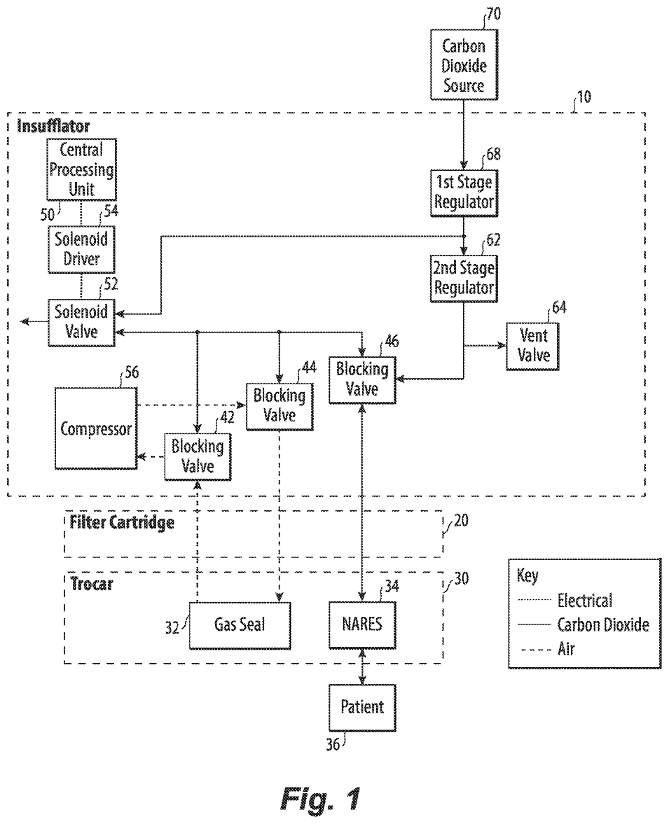

Referring now to the drawings wherein like reference numerals identify similar aspects or features of the subject disclosure, there is illustrated in FIG. 1 a system level schematic diagram of the multi-modal surgical gas delivery device of the subject disclosure, which is designated generally by reference numeral 10. The gas delivery device 10 is of the type disclosed commonly assigned U.S. Pat. No. 9,199,047. It is shown in conjunction with a disposable filter cartridge 20 of the type disclosed in commonly assigned U.S. Pat. No. 8,974,563 and a gas sealed surgical access device or trocar 30 of the type disclosed in commonly assigned U.S. Pat. No. 8,795,223, which includes a gaseous seal zone 32 and a nares 34 for communicating with the abdominal cavity of a patient 36. In this schematic diagram, electrical flow paths, insufflation gas flow paths and recirculation air flow paths are each shown with respective indicator lines.

Referring to FIG. 1, the gas delivery device 10 includes a blocking valve assembly which is depicted as three independent pneumatically actuated blocking valves 42, 44 and 46. Each blocking valve is in pneumatic communication with a vented solenoid valve 52 by way of an independent gas flow path. The solenoid valve 52 is in electrical communication with a solenoid driver 54, which communicates electrically with a central processing unit (CPU) 50 of the gas delivery device 10. During a surgical procedure, the CPU 50 commands the driver 54 to open the solenoid valve 52, which pneumatically actuates the blocking valves 42, 44 and 46, moving them from a blocking position to an operational position relative to the filter cartridge 20, as discussed in more detail below.

It is envisioned and well within the scope of the subject disclosure that a flow control valve other than a vented solenoid valve could be employed in the blocking valve assembly including, for example, a linear actuator driven valve to pilot the blocking valves 42, 44 and 46. Such a linear actuator driven valve could include a linear drive screw driven by a stepper motor to provide precise proportional flow control. Alternatively, a precision gear set could be used as a drive mechanism.

With continuing reference to FIG. 1, blocking valves 42 and 44 are in fluid communication with a compressor 56. More particularly, blocking valve 42 is in fluid communication with the inlet or suction side of compressor 56, while blocking valve 44 is in fluid communication with the outlet or high pressure side of the compressor 56. Together with the filter cartridge 20, these components of the blocking valve assembly form the gas delivery path and gas return path of the gas recirculation circuit of the gas delivery device 10, which functions to generate a gaseous seal 32 within the access device 30.

Blocking valve 46 is in fluid communication with a secondary pressure regulator 62, which together with filter cartridge 20, form the insufflation flow path to the nares 34 of access device 30. The secondary pressure regulator 62 communicates with an emergency vent valve 64 and a primary pressure regulator 66, which receives insufflation gas from a gas source 70, which may be house gas or a supply tank.

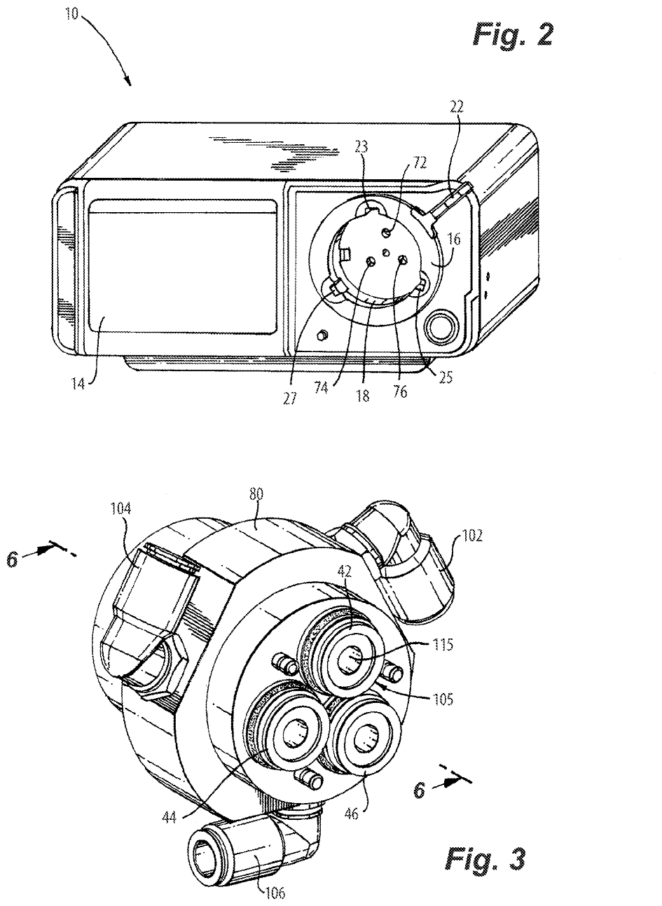

Referring now to FIG. 2, there illustrated the gas delivery device 10 depicted in the schematic diagram of FIG. 1. The gas delivery device 10 includes a housing 12 that includes a graphical user interface screen 14 and a filter cartridge interface 16. The interface 16 defines a reception cavity 18 that is adapted and configured to receive the disposable the filter cartridge 20, which is not shown in this figure.

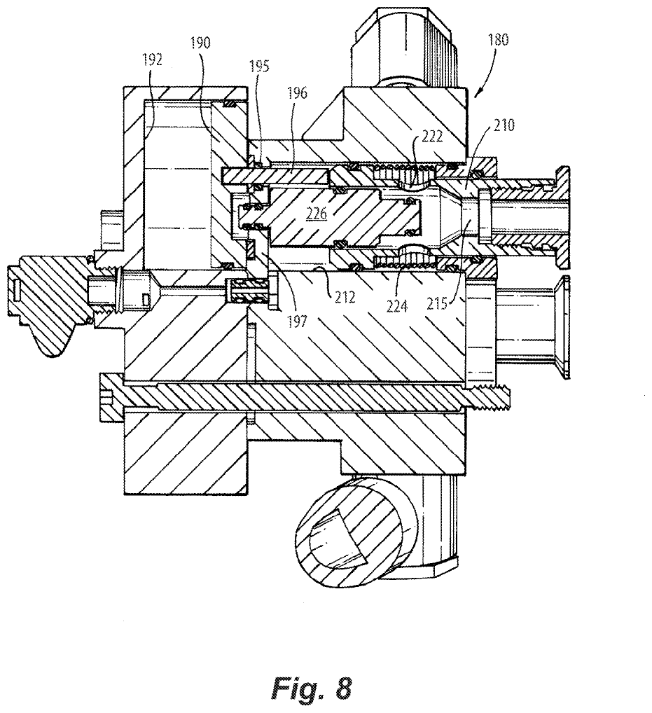

The cartridge interface 16 also includes a latching mechanism having a rotatable lever arm 22 for mechanically seating the filter cartridge 20 in the reception cavity 18 by way of a camming mechanism. More particularly, the rear sealing face of filter cartridge 20 is moved towards the deepest face of the reception cavity 18 by the rotation of a lever arm 22 and subsequent motion of camming features 23, 25 and 27 relative to three corresponding keyed lugs provided on the housing of the filter cartridge 20, as shown in commonly assigned U.S. Pat. No. 9,199,047 (see FIG. 8).

The reception cavity 18 has a three gas ports formed therein, including gas ports 72, 74 and 76, which communicate with respective flow passages extending through the filter cartridge 20.

Referring to FIG. 3, there is illustrated the main valve body of the blocking valve assembly of the subject disclosure, which is designated generally by reference numeral 80. The main valve body 80 houses blocking valves 42, 44 and 46, which were shown schematically in FIG. 1. The main valve body 80 further includes three inlet ports 102, 104 and 106 which define the pathway for gas to flow to the respective blocking valves 42, 44 and 46 housed within the main valve body 80.

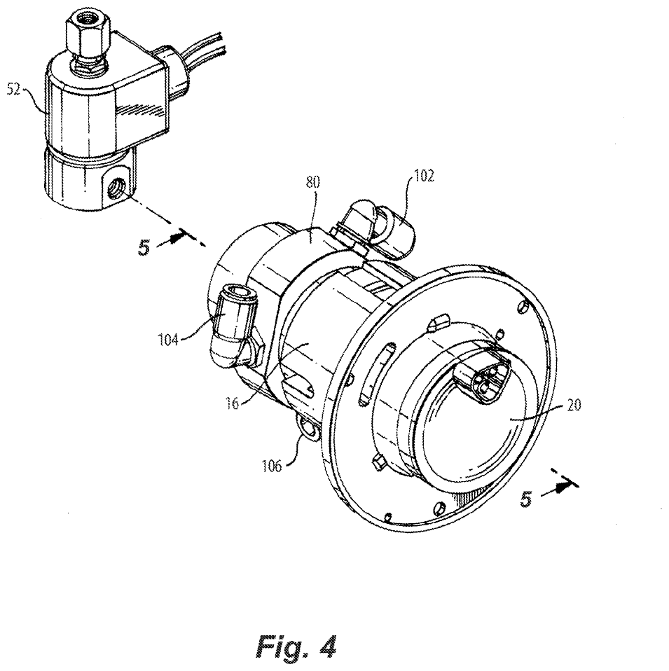

Referring now to FIG. 4, there is illustrated the blocking valve assembly of the subject disclosure, which includes the main valve body 80 adjacent the filter interface 18 of gas delivery device 10 and the vented solenoid valve 52, which was previously shown schematically in FIG. 1. Also shown here in FIG. 4 is the filter cartridge 20 seated within the filter interface 16.

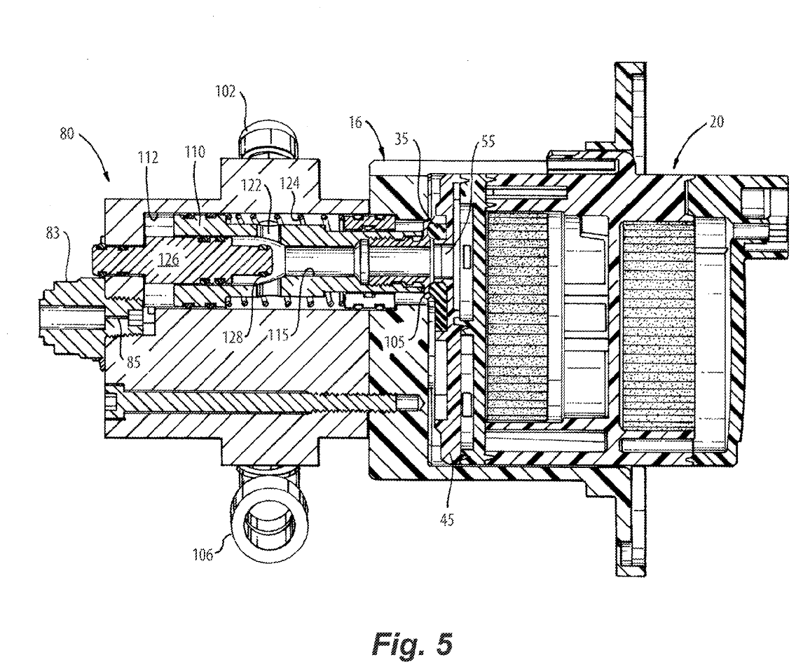

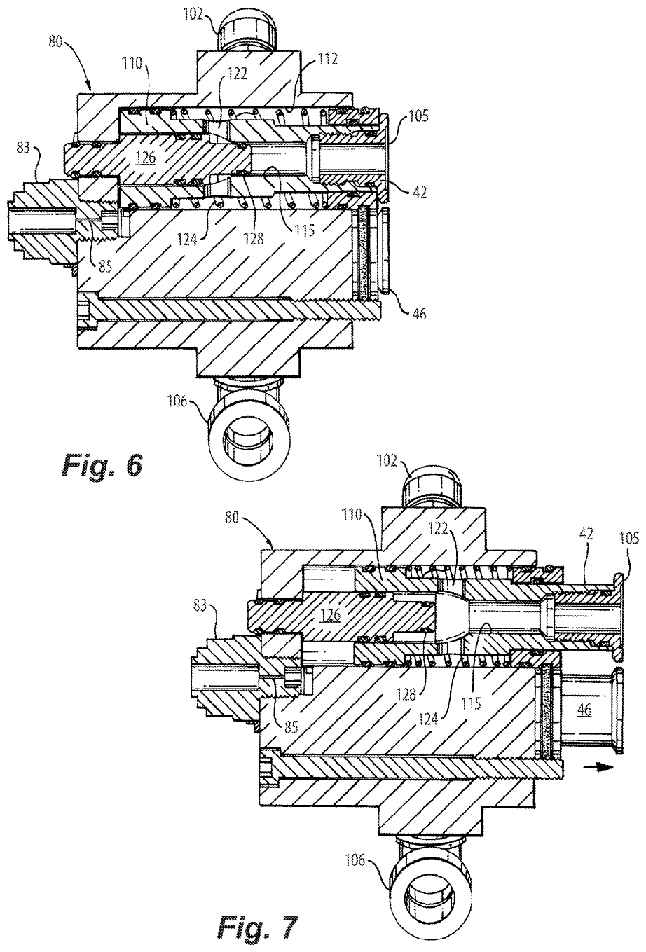

Referring now to FIG. 5, there is illustrated several exemplary internal components of the main valve body 80 of the blocking valve assembly of the subject disclosure. In particular, there is illustrated a pneumatically actuated piston 110 supported for longitudinal translation within a corresponding piston cylinder 112 between a retracted or closed position shown in FIG. 6 and a fully extended or open position shown in FIG. 7, which is shown in the absence of a filter cartridge 20. Spaced pairs of front and rear piston seals are provided on piston 110 to seal between the outer surfaces of the piston 110 and the inner surface of the piston cylinder 112. In an exemplary embodiment, total piston travel from a fully retracted positon to a fully extended is about one-half inch. In FIG. 5, the piston 110 is shown in a partially extended operational position relative to the seated filter cartridge 20, where it resides during a surgical procedure.

The piston 110 has a forward facing sealing face 105, which is best illustrated in FIG. 3. The piston 110 has a central lumen 115, which extends through the sealing face 105. The central lumen 115 is positioned to align with a sealed port 55 of a corresponding planar sealing face 35 located on the rear end of cap 45 of filter cartridge 20 (see, e.g., three co-planar sealing faces located on the rear end of cap of filter the filter cartridge shown in commonly assigned U.S. Pat. No. 8,974,563, incorporated here by reference).

The piston cylinder 112 communicates with inlet port 102 by way of a cross flow path 122. A return spring 124 within the piston cylinder 112 biases the piston 110 into the retraced position shown in FIG. 6. The main valve body 80 includes a rear fitting 83 having a central orifice 85 that communicates with the vented solenoid valve 52 and with the piston cylinder 112 to provide pneumatic pressure thereto. The pressure serves to move the piston 110 to the partially extended operational position shown in FIG. 5 when the driver 54 is commanded to actuate the solenoid valve 60, thereby facilitation the performance of a surgical procedure.

A stationary plug 126 closes off the rear end of piston cylinder 112 and a seal 128 is provided at the distal end of the plug 126 to close off the central lumen 115 of the piston 110 when the piston 110 is in a fully retracted position shown in FIG. 6. In this retracted position, under the bias of return spring 124, the cross flow port 122 is effectively blocked from communicating with central lumen 115 of the piston 110. Thus, gas cannot flow to the filter interface 16 of the gas delivery deice 10. In this position, the gas delivery device 10 can perform a system leak test prior to a surgical procedure, with the filter cartridge 20 seated in reception port 18 of the interface 16. This is inapposite of the blocking valves disclosed in U.S. Pat. No. 9,199,047. A spaced pair of piston seals is provided on the proximal body portion of plug 126 to seal between the outer surfaces of the plug 126 and the inner surface of the central lumen 115 of piston 110.

To perform a surgical procedure, the vented solenoid valve 52 is commanded to open so that pneumatic pressure is delivered to the piston cylinder 112 through the inlet orifice 85 of fitting 83. The inlet orifice 85 is preferably sized to limit flow and thereby reduce patient hazards in the event of dynamic seal failure. Those skilled in the art should readily appreciate that the advantages of the flow restriction provided by the inlet orifice 85 of fitting 83 could be provided by an orifice of the pilot valve used to actuate the pistons of the blocking valves.

When pneumatic pressure is delivered to the piston cylinder 112, the pressure causes piston 110 to translate distally against the bias of return spring 124. As piston 110 moves relative to its corresponding plug 126, the seal 128 opens the central lumen 115 of piston 110.

When opened, gas from inlet port 102 flows into the central lumen 115 of piston 110 by way of cross port 122. The gas then flows to a corresponding sealed port 55 in the rear end cap 45 of filter cartridge 20. In an exemplary embodiment of the subject disclosure, when pneumatic pressure, exceeds approximately 2 bar, the piston 110 move towards the filter cartridge 20, stopping when the piston face 105 contacts the filter cartridge seal 35. When pneumatic pressure is reduced, return spring 124 moves the piston 110 to its retracted and sealed position.

In an exemplary embodiment, the pneumatic gas source for actuating the piston 110 is intermediate pressure carbon dioxide, but in other embodiments, pneumatic pressure could be provided form a different source, or pressurized air it could be provided from the outlet of the compressor 56.

It should be understood by those skilled in the art that the two other pneumatically actuated pistons of blocking valves 44 and 46, which are not shown in FIGS. 5-7, have the same construction as piston 110 of blocking valve 42 and all of the piston cylinders are in pneumatic communication with the central orifice 85 of the main valve body 80. Alternatively, each piston cylinder can have a separate inlet orifice to meter gas flow to the pistons.

Referring now to FIG. 8, there is illustrated another embodiment of the main valve body of the blocking valve assembly of the subject disclosure, which is designated generally by reference numeral 180 and includes three pneumatically actuated two-stage pistons, only one of which is shown in detail herein. As explained in more detail below, the pneumatically actuated two-stage piston configuration provides increased compression force against the three co-planar filter cartridge seals, as compared to the single stage piston 110 in main valve body 80 described above.

The two-stage piston of main valve body 180 includes a proximal drive piston 190 supported for movement within a proximal piston chamber 192 that communicates with a source of pneumatic pressure, by way of a control valve. A seal ring is provided on drive piston 190 to seal between the outer surface of drive piston 190 and the inner wall of the piston chamber 192. Three dowel pins 196, one of which is shown in FIG. 8, extend from the front face of drive piston 190. The dowel pins 196 extends through sealed apertures 195 in the rear wall 197 of piston cylinder 212 to contact the proximal end surface of blocking piston 210. As is the previous embodiment, the blocking piston 210 is mounted for longitudinal translation within piston cylinder 212 against the bias of coiled return spring 224 and relative to stationary plug 226 that seals the central lumen 215 of the piston 210.

In use, to perform a surgical procedure, pneumatic pressure is delivered to piston chamber 192, driving piston 190 forward and causing the dowel pins 196 to urge the blocking piston 210 forward relative to the stationary plug 216. This movement permits gas to flow into the central lumen 215 of piston 210 through cross flow port 222. In the absence of pneumatic pressure, the return spring 224 biases the blocking piston 210 and, in turn, the drive piston 190 to a fully retracted position in the main valve body 180. At such a time, the gas delivery device 10 can perform a leak test with a filter cartridge 20 installed therein.

In sum, the disclosure comprises a plurality of electrically controlled, pneumatically driven pistons translating generally in the direction of a filter cartridge which in their extended state create unobstructed conduits for gas flow to individual lumens within a filter cartridge and in their retracted state seal the gas conduits from the external environment including the filter cartridge ports.

While the subject disclosure has been shown and described with reference to preferred embodiments, those skilled in the art will readily appreciate that changes and/or modifications may be made thereto without departing from the scope of the subject disclosure.

What is claimed is:

1. A blocking valve assembly for a surgical gas delivery device, comprising:
a valve body supporting a plurality of pneumatically actuated pistons, each piston mounted for independent movement within a respective piston cylinder between a first position blocking a respective gas passage communicating with a filter cartridge to facilitate a leak test of the gas delivery device and a second position permitting gas flow through the gas passage communicating with the filter cartridge to facilitate a surgical procedure.

2. A blocking valve assembly as recited in claim 1, wherein the first position of the piston is a retracted position within the piston cylinder and the second position of the piston is an extended position within the piston cylinder.

3. A blocking valve assembly as recited in claim 1, further comprising a control valve in fluid communication with each piston cylinder of the valve body for pneumatically actuating each piston to move each piston from the first position toward the second positon.

4. A blocking valve assembly as recited in claim 3, wherein the control valve is a vented solenoid valve or a motor driven linear actuation valve.

5. A blocking valve assembly as recited in claim 2, wherein each pneumatically actuated piston is biased into the retracted position by a respective coiled return spring.

6. A blocking valve assembly as recited in claim 5, wherein each pneumatically actuated piston is adapted and configured to move toward the extended position, against the bias of the return spring, when pneumatic pressure in the respective piston cylinder exceeds approximately 2 bar.

7. A blocking valve assembly as recited in claim 1, wherein the plurality of pneumatically actuated pistons supported in the valve body includes a first piston corresponding to a gas supply passage of the filter cartridge, a second piston corresponding to a gas return passage of the filter cartridge and a third piston corresponding to an insufflation passage of the filter cartridge.

8. A blocking valve assembly as recited in claim 2, wherein each respective gas passage is defined at least in part by a central lumen of each pneumatically actuated piston.

9. A blocking valve assembly as recited in claim 8, wherein a stationary plug is positioned within each piston cylinder for sealing the central lumen of the corresponding pneumatically actuated piston when the piston is in the retracted position.

10. A blocking valve assembly as recited in claim 1, wherein each pneumatically actuated piston is part of a two-stage piston arrangement, which includes a proximal drive piston for increasing compression force relative to the filter cartridge.

11. A surgical gas delivery device, comprising:
a) a device housing defining a reception cavity configured to receive a filter cartridge having a plurality of gas passages extending therethrough;
b) a valve body located within the device housing adjacent the reception cavity and supporting a plurality of pneumatically actuated pistons, each piston mounted for independent movement within a respective piston cylinder of the valve body between a retracted position blocking a respective gas passage communicating with a filter cartridge to facilitate a leak test of the gas delivery device and an extended position permitting gas flow through the gas passage communicating with the filter cartridge to facilitate a surgical procedure.

12. A surgical gas delivery device as recited in claim 11, further comprising a control valve located within the device housing and in fluid communication with each piston cylinder of the valve body for pneumatically actuating each piston.

13. A surgical gas delivery device as recited in claim 12, wherein the control valve is a vented solenoid valve or a motor driven linear actuation valve.

14. A surgical gas delivery device as recited in claim 11, wherein each pneumatically actuated piston is biased into the retracted position by a respective coiled return spring.

15. A surgical gas delivery device as recited in claim 11, wherein each pneumatically actuated piston is adapted and configured to move to the extended position when pneumatic pressure in the respective piston cylinder exceeds approximately 2 bar.

16. A surgical gas delivery device as recited in claim 11, wherein the plurality of pneumatically actuated pistons supported in the valve body includes a first piston corresponding to a gas supply passage of the filter cartridge, a second piston corresponding to a gas return passage of the filter cartridge and a third piston corresponding to an insufflation passage of the filter cartridge.

17. A blocking valve assembly as recited in claim 11, wherein each respective gas passage is defined at least in part by a central lumen of each pneumatically actuated piston.

18. A blocking valve assembly as recited in claim 17, wherein a stationary plug is positioned within each piston cylinder for sealing the central lumen of the corresponding pneumatically actuated piston when the piston is in the retracted position.

19. A surgical gas delivery device as recited in claim 12, further comprising a driver for activating the control valve to deliver pressurized gas to the valve body to move each of the pneumatically actuated pistons to the extended position, and a processor for commanding the driver to activate the control valve.

20. A blocking valve assembly as recited in claim 11, wherein each pneumatically actuated piston is part of a two-stage piston arrangement, which includes a proximal drive piston for increasing compression force relative to the filter cartridge.
